(19) United States
(12) Patent Application Publication
(10) Pub. No.: US 2007/0190635 A1
(43) Pub. Date: Aug. 16, 2007

(54) Percussion mechanism for a gene gun

(76) Inventor: Hai-Lung Huang, Tainan (TW)

Correspondence Address: Thomas E. Sisson;Jackson Walker, LLP, Suite 2100, 112 E. Pecan, San Antonio, TX, 78205, US

(21) Appl. No.: 11/353,290

(22) Filed: Feb. 10, 2006

(51) Int. Cl. C12M 3/00

(52) U.S. Cl. 435/283.1; 435/285.3

(57) ABSTRACT

A percussion mechanism for a gene gun has a gun body, a percussion assembly and a trigger assembly. The gun body has a trigger chamber, a tubular channel having a valve seat and communicating with the trigger chamber. The percussion assembly is mounted in the tubular channel and has a drive shaft selectively closing the valve seat, an adjusting plug, a spring and a drive lever abutting the drive shaft and extending into the trigger chamber. The trigger assembly is mounted in the trigger chamber and has a trigger and a hammer. When the trigger is pulled, the hammer strikes and pivots the drive lever to pull the drive shaft away from the valve seat, and pressurized gas in the tubular channel discharges into a barrel of the gene gun. The adjusting plug is movable to adjust the spring resilience to control how long the drive shaft is separated from the valve seat.

### BACKGROUND OF THE INVENTION

1. Field of the Invention

The present invention relaters to a gene gun, and more particularly to a percussion mechanism for a gene gun.

2. Description of the Related Art

A gene gun is used in biolistic transformation (i.e. transgenic planting, DNA vaccines and gene therapy). DNA- or vaccine-coated metal (i.e. gold and tungsten) particles are loaded into the gene gun and are injected into any target cell or tissue with a gene gun operating on a pressurized gas (i.e. helium and nitrogen). DNA or vaccines are coated on metal particles and injected into cells so that the cells do not have to be removed from tissue to achieve the desired effect.

However, conventional gene guns do not have enough sustained force. When the gene gun is activated, a pressurized gas pulse propels the DNA- or vaccine-coated particles for a very short time. Consequently, the DNA or vaccine tends to stay in cutis and fails to achieve the desired effect.

### SUMMARY OF THE INVENTION

The primary objective of the present invention is to provide a percussion mechanism for a gene gun to prolong the time that pressurized gas in the gene gun propels DNA- or vaccine-coated metal particles and effectively deliver the DNA- or vaccine-coated metal particles to any target cell or tissue.

The percussion mechanism in accordance with the present invention for a gene gun having a barrel is attached to the barrel and has a gun body, a percussion assembly and a trigger assembly.

A percussion mechanism for a gene gun has a gun body, a percussion assembly and a trigger assembly. The gun body has a trigger chamber, a tubular channel having a valve seat. The percussion assembly is mounted in the tubular channel and has a drive shaft selectively closing the valve seat, an adjusting plug, a spring and a drive lever abutting the drive shaft and extending into the trigger chamber. The trigger assembly is mounted in the trigger chamber and has a trigger and a hammer. When the trigger is pulled, the hammer strikes and pivots the drive lever to pull the drive shaft away from the valve seat, and pressurized gas in the tubular channel discharges into a barrel of the gene gun. The adjusting plug is movable to adjust the spring resilience to control how long the drive shaft is separated from the valve seat.

drive lever Because the resilience of the spring of the percussion assembly is adjustable, the time that the distal end of the drive shaft is away from the valve seat can be adjusted so the pressurized gas in pressure chamber has a longer time to push the DNA- or vaccine-coated metal particles in the barrel. Thus, the DNA- or vaccine-coated metal particles can be pushed more powerfully to ensure penetration of the cutis and be delivered into any target cell or tissue to achieve the expected effect.

Other objectives, advantages and novel features of the invention will become more apparent from the following detailed description when taken in conjunction with the accompanying drawings.

### BRIEF DESCRIPTION OF THE DRAWINGS

FIG. 1 is a side view in partial section of a first embodiment of a percussion mechanism for a gene gun in accordance with the present invention;

FIG. 2 is an operational side view in partial section of the percussion mechanism in FIG. 1 in a cocked position;

FIG. 3 is an operational side view in partial section of the percussion mechanism in FIG. 2 with the trigger being pulled;

FIG. 4 is an operational side view in partial section of the percussion mechanism in FIG. 3 with the hammer moving the drive shaft back and opening the valve seat;

FIG. 5 is an operational side view in partial section of the percussion mechanism in FIG. 4 with the hammer providing a resistance to the drive shafty's forward movement;

FIG. 6 is an operational side view in partial section of the percussion mechanism in FIG. 3 with the drive shaft pressed forward by the drive shaft spring and closing the valve seat;

FIG. 7 is an operational side view in partial section-of the percussion mechanism in FIG. 1 with the adjusting plug being rotated to adjust the drive shaft spring pressure on the drive shaft;

FIG. 8 is an operational side view in partial section of a second embodiment of a percussion mechanism in accordance with the present invention for a gene gun in a cocked position;

FIG. 9 is an operational side view in partial section of the percussion mechanism in FIG. 8 with the trigger being pulled;

FIG. 10 is an operational side view in partial section of the percussion mechanism in FIG. 9 with the hammer pressing the drive shaft back and opening the valve seat; and

FIG. 11 is an operational side view in partial section of the percussion mechanism in FIG. 10 with the drive shaft spring pressing the drive shaft forward to close the valve seat and the hammer back to a cocked position.

### DETAILED DESCRIPTION OF THE PREFERRED EMBODIMENT

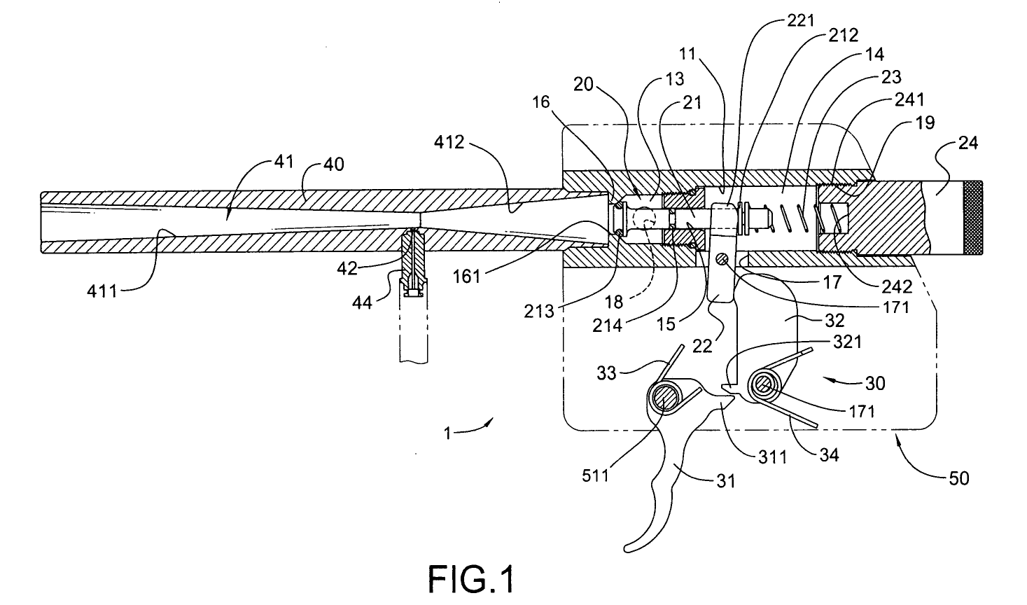

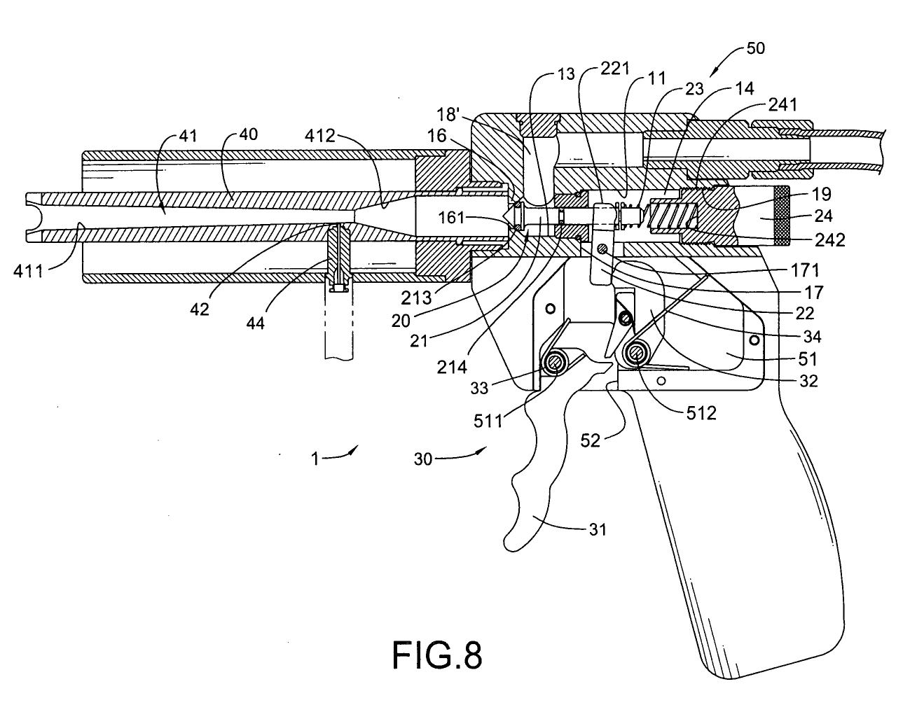

With reference to FIGS. 1 and 8, a percussion mechanism (1) in accordance with the present invention for a gene gun comprising a percussion mechanism (1) and a barrel (40) is connected to an external source (not shown) of propellant gas through a flexible tube (not numbered), pressurizes the gas, ejects the compressed gas into the barrel (40) and has a gun body (50), a percussion assembly (20) and a trigger assembly (30).

The gun body (50) has two sides (not numbered), a rear (not numbered), an outside surface (not numbered), a trigger chamber (51), a trigger hole (52), a tubular channel (11), a pivot hole (17) and a gas inlet port (18, 18′).

The trigger chamber (51) is defined in the gun body (50) and has an inner surface (not numbered), an optional trigger spring mount (511) and an optional hammer spring mount (512). The trigger spring mount (511) and the hammer spring mount (512) are formed on the inner surface of the trigger chamber (51).

The trigger hole (52) is defined through the outside surface of the gun body (50) and communicates with the trigger chamber (51).

The tubular channel (11) is defined longitudinally in the gun body (50) above the trigger chamber (51) and has a front end (not numbered), a rear end (not numbered), an inner surface (not numbered), a valve seat (16) and an internal thread (19). The valve seat (16) is formed on and protrudes radially in from the inner surface of the tubular channel (11) near the front end of the tubular channel (11) and has a central through hole (161). The central through hole (161) is defined coaxially through the valve seat (16) and has an optional tapered inner edge (not numbered). The tapered inner edge faces the rear end of the tubular channel (11). The internal thread (19) is formed on the inner surface of the tubular channel (11) near the rear end of the tubular channel (11).

The pivot hole (17) is formed in the gun body (50), communicates with the tubular channel (11) and the trigger chamber (51) and has an inner surface (not numbered) and an optional pivot pin (171). The pivot pin (171) is formed on the inner surface of the pivot hole (17).

The gas inlet port (18, 18′) is formed through the outside surface of the gun body (50), may be formed through one of the two sides of the gun body (50), may be formed through the rear of the gun body (50), communicates with the tubular channel (11), may form an L-shaped channel (not numbered) and is connected to an external gas supply tube (not shown) through which pressurized gas flows into the tubular channel (11).

The percussion assembly (20) is mounted in the tubular channel (11) in the gun body (50) and has a sleeve (15), a pressure chamber (13), an adjusting plug (24), an actuating chamber (14), a drive shaft (21), a drive lever (22) and a drive shaft spring (23).

The sleeve (15) is mounted with an airtight fit in the tubular channel (11) in the gun body (50) and has a central guide hole (not numbered). The central guide hole is formed axially through the sleeve (15).

The pressure chamber (13) is formed in the tubular channel (11) between the valve seat (16) and the sleeve (15) and communicates with the gas inlet port (18. 18′).

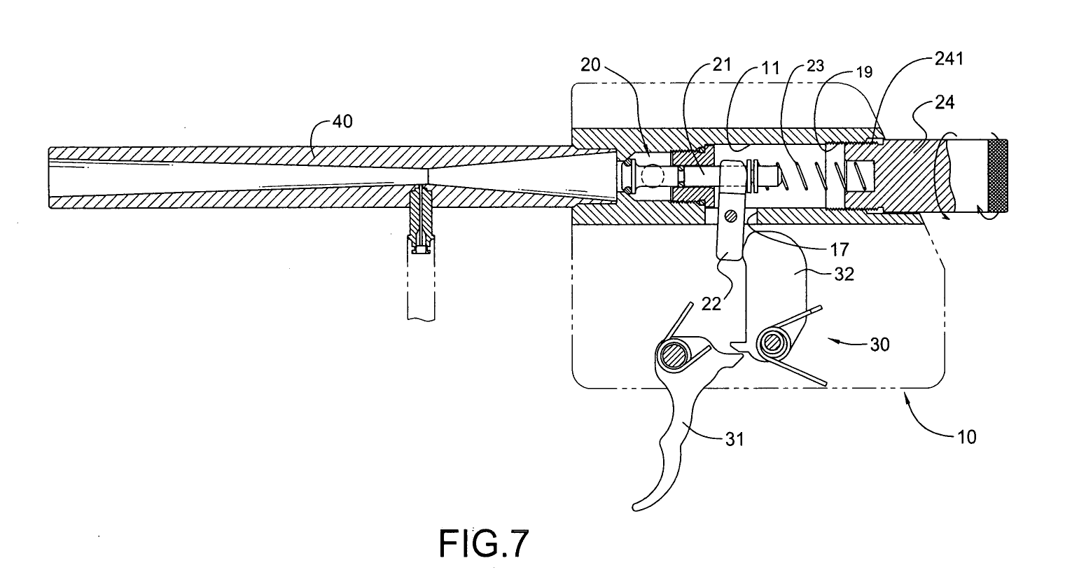

The adjusting plug (24) is mounted adjustable in the rear end of the tubular channel (11) and has an inner end (not numbered), an outer thread (241) and an optional spring mounting hole (242). The outer thread (241) is formed around the adjusting plug (24) at the inner end and screws onto the internal thread (19) near the rear end of the tubular channel (11). With further reference to FIG. 7, the adjusting plug (24) is turned to move into or out of the tubular channel (11). The spring mounting hole (242) is defined in the inner end of the adjusting plug (24).

The actuating chamber (14) is formed in the tubular chamber (11) between the sleeve (15) and the adjusting plug (24) and communicates with the pivot hole (17).

The drive shaft (21) is mounted coaxially in the tubular channel (11) to selectively close the valve seat (16) and slidably through the central guide hole in the sleeve (15) with an airtight fit and has a front end (not numbered), a rear end (not numbered), an annular flange (212), a valve disk (not numbered), an optional valve disk O-ring (213), an optional annular sleeve groove (not numbered) and an optional sleeve O-ring (214). The annular flange (212) is formed around and protrudes radially out from the drive shaft (21) near the rear end. The valve disk is formed around and protrudes out from the front end of the drive shaft (21), corresponds to the central through hole (161) in the valve seat (16), selectively closes the central through hole (161) and has an outer edge and an optional annular valve disk groove (not numbered). The annular valve disk groove is formed around the outer edge of the valve disk, corresponds to the central through hole (161) in the valve seat (16) and selectively abuts the valve seat (16) to close the central through hole (161) in the valve seat (16). The valve disk O-ring (213) is mounted in the annular valve disk groove to abut the tapered inner edge of the central through hole (161) and close the valve seat (16). The annular sleeve groove is defined around the drive shaft (21). The sleeve O-ring (214) is mounted in the annular sleeve groove to form an airtight fit in the central guide hole of the sleeve (15).

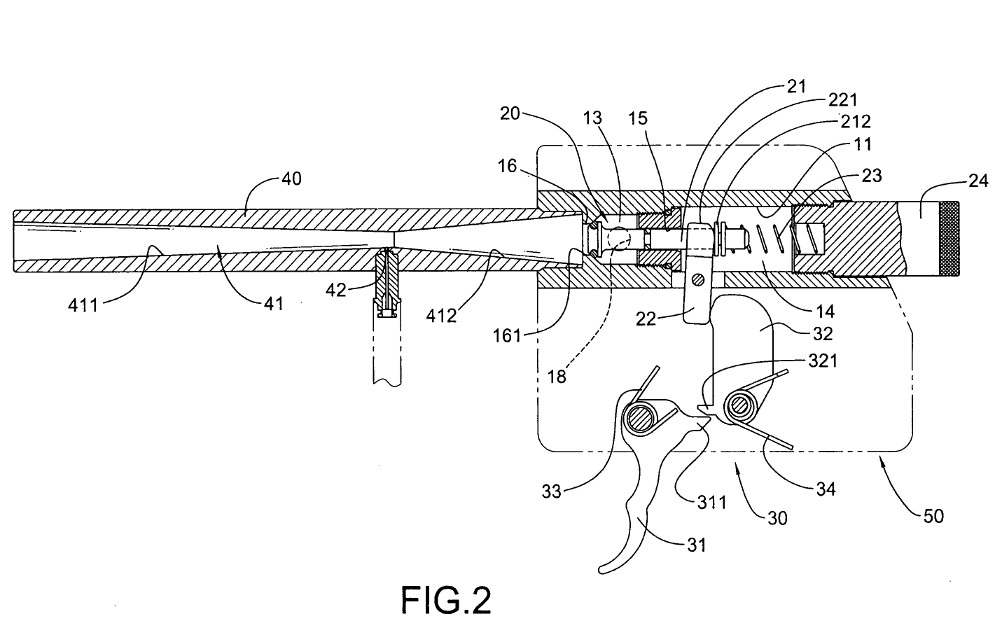

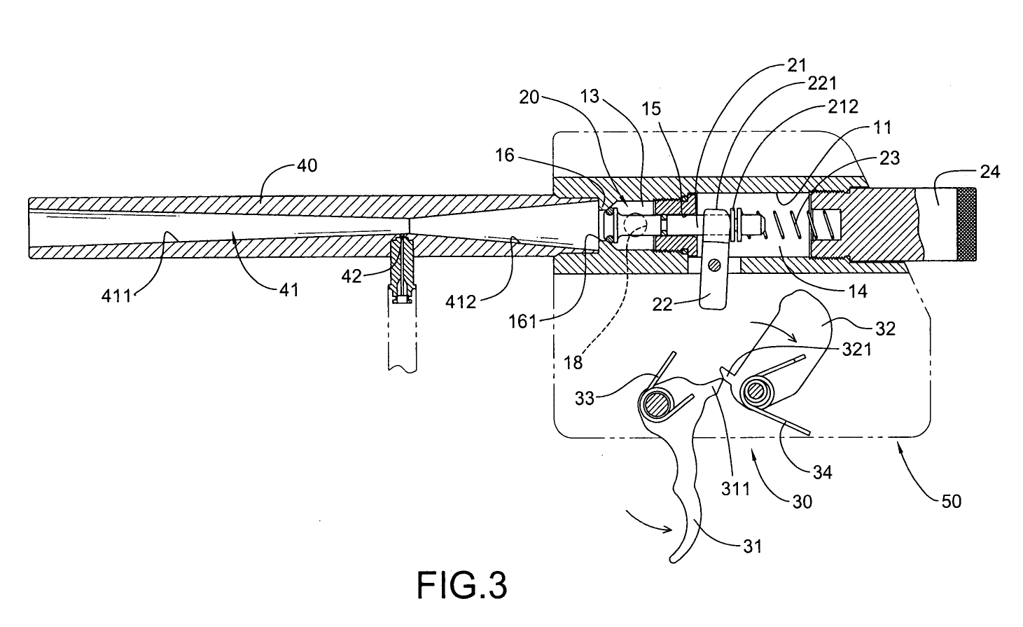

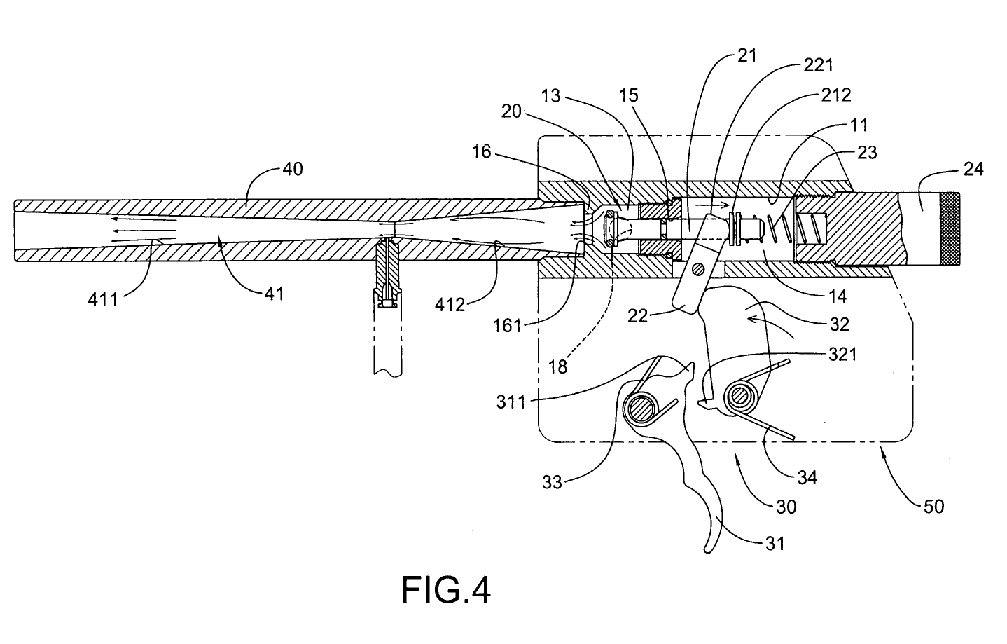

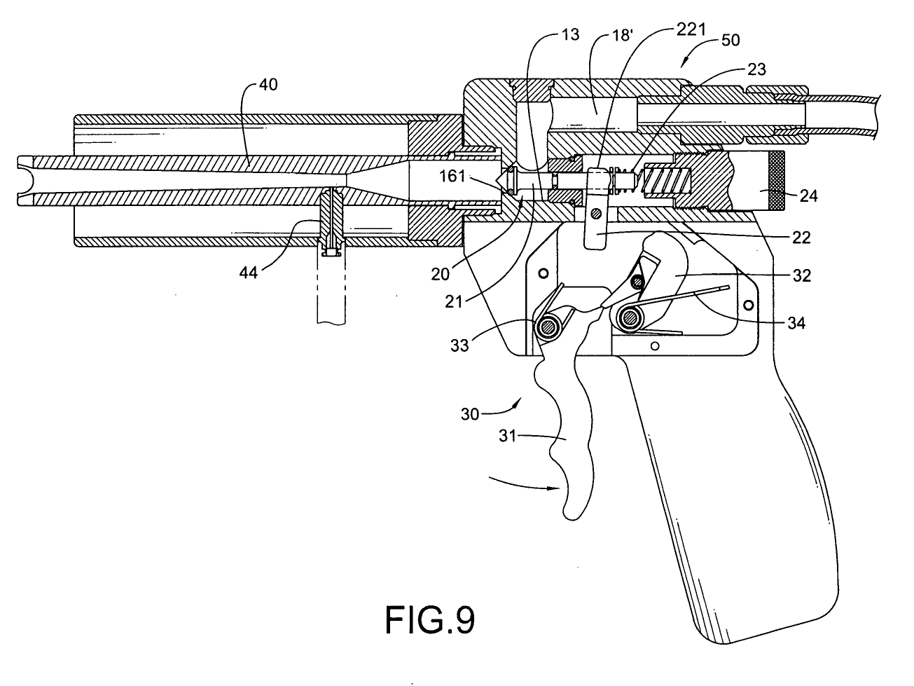

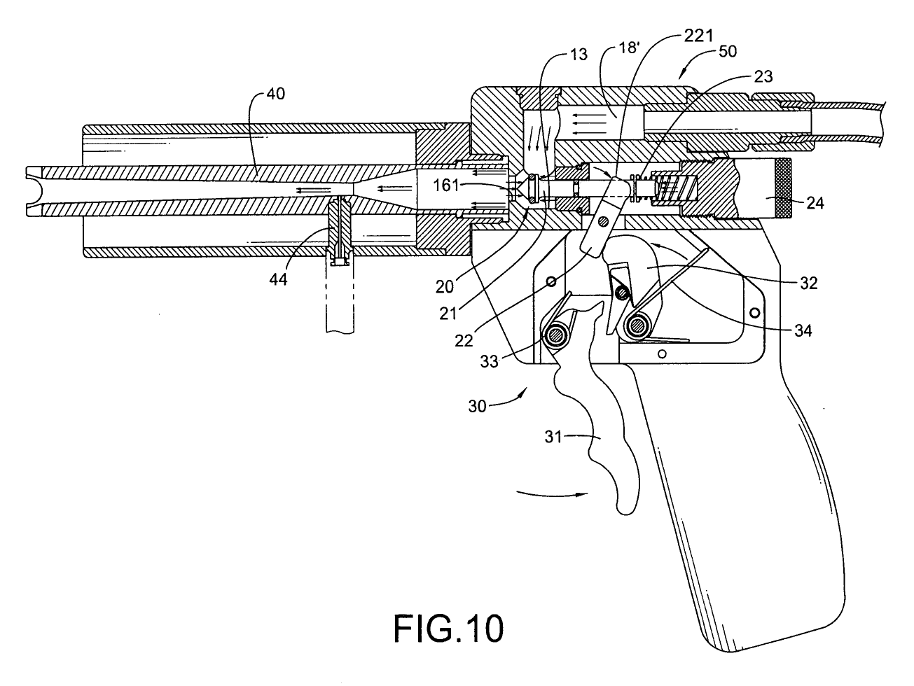

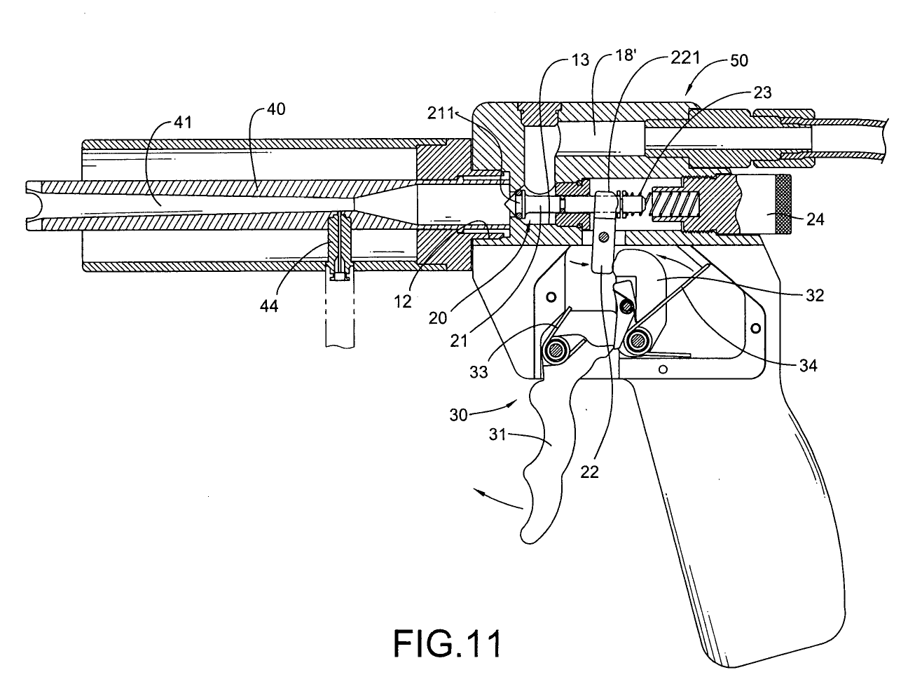

The drive lever (22) is mounted pivotally in and protruding from the pivot hole (17) in the gun body (50) and has an inner end (not numbered), an outer end (not numbered), an optional pivot hole (not numbered) and two optional tabs (221). With further reference to FIGS. 2-4 and 9-11, the inner end of the drive lever (22) extends into the actuating chamber (14) and abuts the annular flange (212) to selectively pull the valve disk of the drive shaft (21) away from the central through hole (161) in the valve seat (16) and allow pressurized gas in the pressure chamber (13) to discharge through the central through hole (161). The outer end of the drive lever (22) extends into the trigger chamber (51) in the gun body (50). The pivot hole is defined through the drive lever (22) between the inner and outer ends and is mounted pivotally around the pivot pin (171) in the pivot hole (17) in the gun body (50) to allow the drive lever (22) to pivot in the pivot hole (17). The two tabs (221) are formed diametrically opposite to each other on and protrude longitudinally from the inner end of the drive lever (22), hold the drive shaft (21) and abut the annular flange (212) on the drive shaft (21) to pull the drive shaft (21) smoothly.

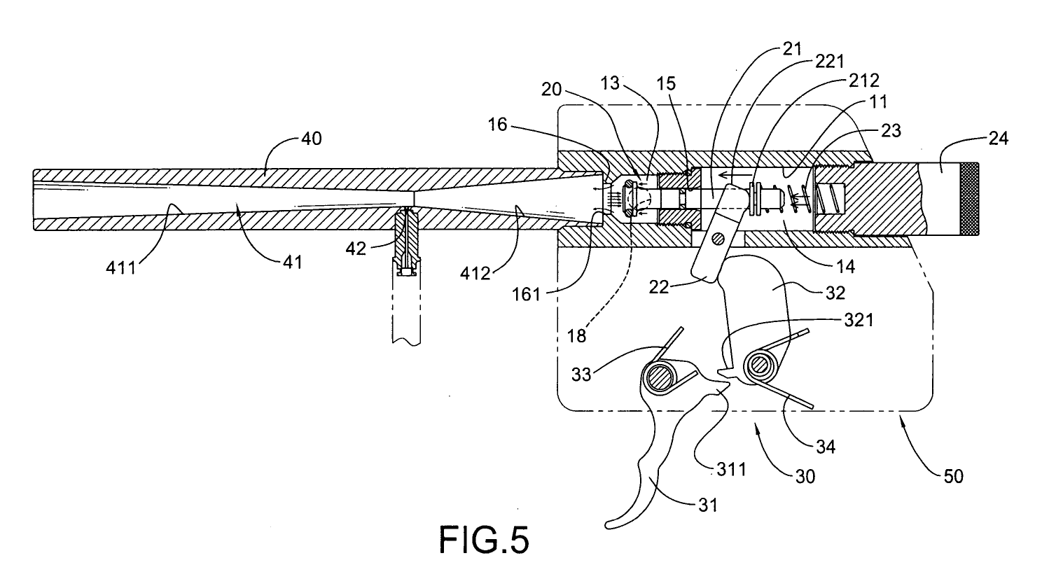

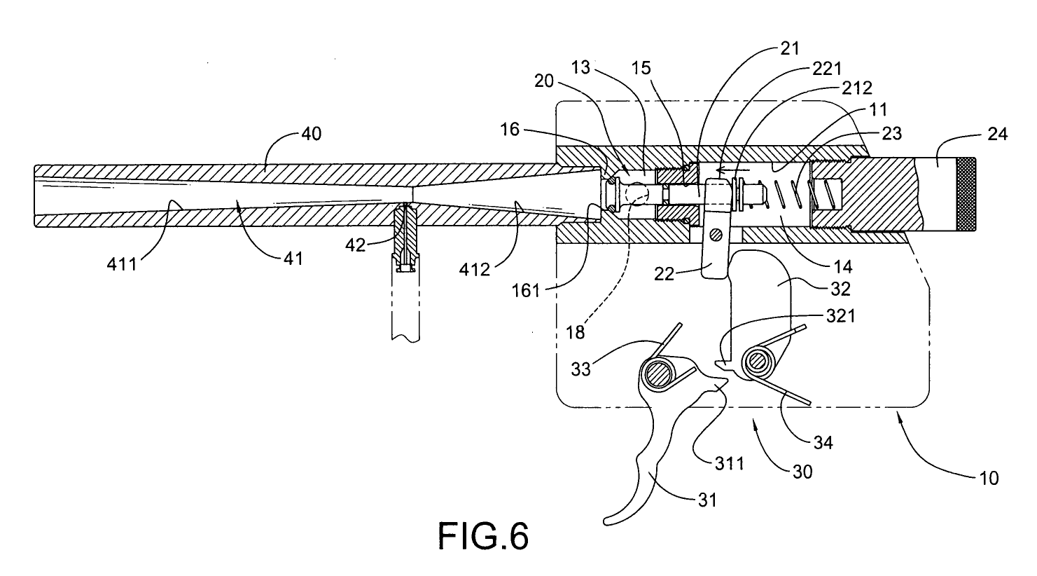

With further reference to FIGS. 5 and 6, the drive shaft spring (23) is mounted between the rear end of the drive shaft (21) and the adjusting plug (24) to reposition the drive shaft (21) when the drive lever (22) releases the drive shaft (21) and has a resilient force (not numbered), a front end (not numbered) and a rear end (not numbered). With further reference to FIG. 7, the resilient force of the spring (23) is adjusted by screwing the adjusting plug (24) into or out of the tubular channel (11). The rear end is mounted stably in the spring mounting hole (242) in the adjusting plug (24).

The trigger assembly (30) is mounted in the trigger chamber (51) and has a trigger (31), a trigger spring (33), a hammer (32) and a hammer spring (34).

The trigger (31) is mounted pivotally in the trigger chamber (51), protrudes from the trigger hole (52), activates the percussion mechanism (1) when the trigger (31) is squeezed and has an outer end (not numbered), an inner end (not numbered), an optional trigger hook (311) and an optional pivot hole (not numbered). The outer end of the trigger (31) protrudes through the trigger hole (52) and is squeezed to activate the percussion mechanism (1). The inner end is mounted pivotally in the trigger chamber (51). The trigger hook (311) is formed on and protrudes out from the inner end of the trigger (31). The pivot hole is defined through the trigger (31) near the inner end and is mounted pivotally around the trigger spring mount (511) in the trigger chamber (51) to pivot the trigger (31).

The trigger spring (33) is mounted integrally with the trigger (31) around the trigger spring mount (511) in trigger chamber (51), biases the outer end of the trigger (31) forward and repositions the trigger (31) when the trigger (31) is released.

The hammer (32) is mounted pivotally in the trigger chamber (51), pivots the drive lever (22) to pull the drive shaft (21) away from the valve seat (15) and has a proximal end (not numbered), a distal end (not numbered), a proximal end (not numbered), an optional hammer hook (321) and an optional pivot hole (not numbered). The distal end of the hammer (32) abuts the outer end of the drive lever (22) in a default situation. The proximal end of the hammer (32) is mounted pivotally in the trigger chamber (51) near the inner end of the trigger (31), is pressed by the inner end of the trigger (31) to pivot and cock the hammer (32) when the trigger (31) is pulled and drives forward and strikes and pivots the outer end of the drive lever (22), which pulls the valve disk on the drive shaft (21) away from the valve seat (16). The hammer hook (321) is formed on and protrudes from the proximal end of the hammer (32) and is pressed by the trigger hook (311) on the trigger (31) such that the hammer (32) can be rotated by the trigger (31). The pivot hole is defined through the hammer (32) near the proximal end and is mounted pivotally around the hammer spring mount (512) in the trigger chamber (51) to pivot the hammer (32) in the trigger chamber (51).

The hammer spring (34) is mounted integrally with the hammer (32) around the hammer spring mount (512) in the trigger chamber (51) and pivots the hammer (32) when the hammer (32) is cocked and released by the trigger (31).

The barrel (40) is mounted in the front end of the tubular channel (11) in the gun body (50), provides a path for the gas in the pressure chamber (13) and has an outside surface (not numbered), a proximal end (not numbered), a distal end (not numbered), an injection channel (41), a cartridge-mounting hole (not numbered) and a inhaling port (42). The proximal end of the barrel (40) is mounted in the front end of the tubular channel (11). The injection channel (41) is defined axially through the barrel (40) and has an outer tapered channel (411) and an inner tapered channel (412). The outer tapered channel (411) is defined coaxially in the barrel (40) through the distal end of the barrel (40), diverges as the outer tapered channel (411) approaches the distal end of the barrel (40) and has a narrow rear end (not numbered). The inner tapered channel (412) is defined coaxially in the barrel (40) through the proximal end of the barrel (40), diverges as the inner tapered channel (412) approaches the proximal end of the barrel (40) and has a narrow front end (not numbered). The narrow front end of the inner tapered channel (412) is attached to and communicates with narrow rear end of the outer tapered channel (412) and forms a narrowest longitudinal bore (not numbered) in the barrel (40). The cartridge-mounting hole is defined radially in the outside surface of the barrel (40) just forward of the narrowest bore in the barrel (40) and holds a DNA or vaccine cartridge (44). The inhaling port (42) is defined coaxially with the cartridge-mounting hole, communicates with the cartridge-mounting hole and the outer tapered channel (411) near the narrow rear end and allows DNA- or vaccine-coated metal particles in the DNA or vaccine cartridge (44) to be into the injection channel (41) when pressurized gas passes through the injection channel (41) and be delivered into any target cell or tissue.

The most significant advantage and improvement of the percussion mechanism (1) for a gene gun as described is the ability to adjust the resilient force of the drive shaft spring (23), which allows the disk on the drive shaft (22) to stay away from the central through hole (161) in the valve seat (16) longer so pressurized gas in the pressure chamber (13) has longer time to push the DNA- or vaccine-coated metal particles. Consequently, the DNA- or vaccine-coated metal particles pass more easily through the cutis and are delivered into any target cell or tissue to achieve the expected effect.

Even though numerous characteristics and advantages of the present invention have been set forth in the foregoing description, together with details of the structure and features of the invention, the disclosure is illustrative only. Changes may be made in the details, especially in matters of shape, size and arrangement of parts within the principles of the invention to the full extent indicated by the broad general meaning of the terms in which the appended claims are expressed.

What is claimed is:

1. A percussion mechanism for a gene gun comprising:
a gun body having
two sides;
a rear;
an outside surface;
a trigger chamber being defined in the gun body and having an inner surface;
a trigger hole being defined through the outside surface of the gun body and communicating with the trigger chamber;
a tubular channel being defined longitudinally in the gun body above the trigger chamber and having
a front end;
a rear end;
an inner surface;
a valve seat being formed on and protruding radially in from the inner surface of the tubular channel near the front end of the tubular channel and having a central through hole defined coaxially through the valve seat; and
an internal thread being formed on the inner surface of the tubular channel near the rear end of the tubular channel;
a pivot hole being formed in the gun body, communicating with the tubular channel and the trigger chamber and having an inner surface; and
a gas inlet port being formed through the outside surface of the gun body, communicating with the tubular channel;
a percussion assembly being mounted in the tubular channel in the gun body and having
a sleeve being mounted with an airtight fit in the tubular channel in the gun body and having a central guide hole defined axially through the sleeve;
a pressure chamber being formed in the tubular channel between the valve seat and the sleeve and communicating with the gas inlet port;
an adjusting plug being mounted adjustable in the rear end of the tubular channel and having
an inner end; and
an outer thread being formed around the adjusting plug at the inner end and screwing onto the internal thread in the tubular channel;
an actuating chamber being formed in the tubular chamber between the sleeve and the adjusting plug and communicating with the pivot hole;
a drive shaft mounted coaxially in the tubular channel to selectively close the valve seat and slidably through the central guide hole in the sleeve with an airtight fit and having
a front end;
a rear end;
an annular flange being formed around and protruding radially out from the drive shaft near the rear end; and
a valve disk being formed around and protruding out from the front end of the drive shaft, corresponding to the central through hole in the valve seat, selectively closing the central through hole and having an outer edge;
a drive lever being mounted pivotally in and protruding from the pivot hole in the gun body and having
an inner end extending into the actuating chamber and abutting the annular flange; and
an outer end extending into the trigger chamber in the gun body; and
a drive shaft spring being mounted between the rear end of the drive shaft and the adjusting plug and having
a resilient force;
a front end; and
a rear end; and
a trigger assembly being mounted in the trigger chamber and having
a trigger being mounted pivotally in the trigger chamber, protruding from the trigger hole, activating the percussion mechanism when the trigger is squeezed and having
an outer end protruding through the trigger hole and being squeezed to activate the percussion mechanism; and
an inner end being mounted pivotally in the trigger chamber;
a trigger spring being mounted integrally with the trigger in trigger chamber, biasing the outer end of the trigger forward and repositioning the trigger when the trigger is released;
a hammer being mounted pivotally in the trigger chamber, pivoting the drive lever to pull the drive shaft away from the valve seat and having
a distal end abutting the outer end of the drive panel in a default situation; and
a proximal end being mounted pivotally in the trigger chamber near the inner end of the trigger, being pressed by the inner end of the trigger to pivot and cock the hammer when the trigger is pulled and driving forward and striking and pivoting the outer end of the drive lever; and
a hammer spring being mounted integrally with the hammer in the trigger chamber and pivoting the hammer when the hammer is cocked and released by the trigger.

2. The percussion mechanism as claimed in claim 1, wherein the gas inlet port is defined through the outside surface and one of the two sides of the gun body.

3. The percussion mechanism as claimed in claim 1, wherein the gas inlet port is defined though the outside surface and the rear of the gun body.

4. The percussion mechanism as claimed in claim 1, wherein
the central through hole of the valve seat further has a tapered inner edge facing the rear end of the tubular channel; and
the valve disk on the drive shaft further has an annular valve disk groove formed around the outer edge of the valve disk, corresponding to the central through hole in the valve seat and selectively abutting the valve seat to close the central through hole in the valve seat;
the drive shaft further has
a valve disk O-ring mounted in the annular valve disk groove to selectively abut the tapered inner edge of the central through hole in the valve seat and close the valve seat;
an annular sleeve groove formed around the drive shaft; and
a sleeve O-ring mounted in the annular sleeve groove to form an airtight fit in the central guide hole of the sleeve.

5. The percussion mechanism as claimed in claim 1, wherein
the adjusting plug further has a spring mounting hole defined in the inner end of the adjusting plug; and
the rear end of the drive shaft spring is mounted stably in the spring mounting hole in the adjusting plug.

6. The percussion mechanism as claimed in claim 1, wherein the drive lever further has two tabs formed diametrically opposite to each other on and protruding longitudinally from the inner end of the drive lever, holding the drive shaft and abutting the annular flange on the drive shaft.

7. The percussion mechanism as claimed in claim 1, wherein
the trigger further has a trigger hook formed on and protruding out from the inner end of the trigger; and
the hammer further has a hammer hook formed on and protruding from the proximal end of the hammer and being pressed by the trigger hook on the trigger.

8. The percussion mechanism as claimed in claim 1, wherein
the trigger chamber further has a trigger spring mount formed on the inner surface of the trigger chamber;
the trigger further has a pivot hole defined through the trigger near the inner end and mounted pivotally around the trigger spring mount in the trigger chamber; and
the trigger spring is mounted around the trigger spring mount in the trigger chamber.

9. The percussion mechanism as claimed in claim 1, wherein
the trigger chamber further has a hammer spring mount formed on the inner surface of the trigger chamber;
the hammer further has a pivot hole defined through the hammer near the proximal end and mounted pivotally around the trigger spring mount in the trigger chamber; and
the hammer spring is mounted around the hammer spring mount in the trigger chamber.

10. The percussion mechanism as claimed in claim 1, wherein
the pivot hole in the gun body further has a pivot pin formed on the inner surface of the pivot hole; and
the drive lever further has a pivot hole defined through the drive lever between the inner end and outer end of the drive lever and mounted pivotally around the pivot pin in the pivot hole in the gun body.

11. The percussion mechanism as claimed in claim 2, wherein the gas inlet port forms an L-shaped channel.
